(19) United States
(12) Patent Application Publication
(10) Pub. No.: US 2009/0299179 A1
(43) Pub. Date: Dec. 3, 2009

(54) Method For Detecting Cardiac Transplant Rejection

(76) Inventors: Joan Carol Main, Scottsdale, AZ (US); Matt Umland, Anthem, AZ (US); Jamil Tajik, Fountain Valley, AZ (US); Bijoy Khandheria, Fountain Hills, AZ (US); Joan Lusk, Desert Hills, AZ (US)

Correspondence Address: QUARLES & BRADY LLP, 411 E. WISCONSIN AVENUE, SUITE 2040, MILWAUKEE, WI, 53202-4497, US

(21) Appl. No.: 12/476,557

(22) Filed: Jun. 2, 2009

Related U.S. Application Data
(60) Provisional application No. 61/130,645, filed on Jun. 2, 2008.

(51) Int. Cl. A61B 8/00

(52) U.S. Cl. 600/438

(57) ABSTRACT

A method for detecting cardiac rejection in heart transplant patients is disclosed. The wall mechanics, such as wall shear, stress, and strain, of a heart ventricle of the transplant patient are measured. A property of the native vasculature is also measured. The measured property may include the wall mechanics or thickness of the native blood vessel. The wall mechanics measurements of the ventricle and the measured property of the native vasculature are compared, and the comparison is outputted. The outputted comparison is matched either to similarly-obtained comparisons from heart transplant patients undergoing early cardiac rejection of the transplanted heart or to similarly-obtained comparisons from heart transplant patients with normally-functioning hearts.

### CROSS REFERENCE TO RELATED APPLICATIONS

This application is based on, claims the benefit of, and incorporates herein by reference U.S. Provisional Application Ser. No. 61/130,645, filed Jun. 2, 2008, and entitled “Method for Detecting Early Cardiac Transplant Rejection.”

### BACKGROUND OF THE INVENTION

The present invention relates to systems and methods for ultrasound imaging and, more particularly, to a system and method for detecting early cardiac rejection in a heart transplant patient by using an ultrasound echocardiogram to measure and compare the ventricular wall mechanics of the patient's transplanted heart to a measured property of the patient's native vasculature.

The rejection of a transplanted heart by a transplant patient's immune system can be a serious complication in heart transplantation. The transplanted heart contains antigens that can stimulate an immune response from the transplant patient's lymphocytes. Despite routine use of immunosuppressive drugs, the rapidity and severity of heart transplant rejection reactions are often variable and unpredictable, and rejection reactions can be life-threatening.

After heart transplantation, patients are generally monitored for rejection by endomyocardial biopsies that are typically obtained from the right ventricle on a periodic basis. Endomyocardial biopsy is invasive and carries significant risk of adverse side effects. In addition, the results are subject to a high degree of variability, and are not effective in identifying rejections within the first few days of transplantation, possibly delaying needed immunosuppressant therapy. Thus, there is a need in the art for a non-invasive and consistent method for detecting early signs of rejection for transplanted hearts.

Sonography is well-known in the art as a non-invasive method of using ultrasound to image and gathering information about organs and other soft tissues. Echocardiography is sonography of the heart and echocardiograms generally use standard ultrasound techniques to image two-dimensional slices of the heart.

There are a number of modes in which ultrasound can be used to produce images of objects. The ultrasound transmitter may be placed on one side of the object and the sound transmitted through the object to the ultrasound receiver placed on the other side (“transmission mode”). With transmission mode methods, an image may be produced in which the brightness of each pixel is a function of the amplitude of the ultrasound that reaches the receiver (“attenuation” mode), or the brightness of each pixel is a function of the time required for the sound to reach the receiver (“time-of-flight” or “speed of sound” mode). In the alternative, the receiver may be positioned on the same side of the object as the transmitter and an image may be produced in which the brightness of each pixel is a function of the amplitude or time-of-flight of the ultrasound reflected from the object back to the receiver (“refraction”, “backscatter” or “echo” mode). The present invention relates to a backscatter method for producing ultrasound images.

There are a number of well known backscatter methods for acquiring ultrasound data. In the so-called “A-mode” scan method, an ultrasound pulse is directed into the object by the transducer and the amplitude of the reflected sound is recorded over a period of time. The amplitude of the echo signal is proportional to the scattering strength of the refractors in the object and the time delay is proportional to the range of the refractors from the transducer. In the so-called “B-mode” scan method, the transducer transmits a series of ultrasonic pulses as it is scanned across the object along a single axis of motion. The resulting echo signals are recorded as with the A-mode scan method and their amplitude is used to modulate the brightness of pixels on a display. The location of the transducer and the time delay of the received echo signals locates the pixels to be illuminated. With the B-mode scan method, enough data are acquired from which a two-dimensional image of the refractors can be reconstructed. Rather than physically moving the transducer over the subject to perform a scan it is more common to employ an array of transducer elements and electronically move an ultrasonic beam over a region in the subject.

In addition, later ultrasound systems allow real-time 3D imaging in echocardiograms. Using pulsed or continuous wave Doppler ultrasound, an echocardiogram can also produce accurate assessments of the velocity of blood and cardiac tissue at any chosen point. Doppler systems employ an ultrasonic beam to measure the velocity of moving reflectors, such as flowing blood cells or the movement of tissue. Blood velocity or tissue velocity is detected by measuring the Doppler shifts in frequency imparted to ultrasound by reflection from moving red blood cells. Accuracy in detecting the Doppler shift at a particular point depends on defining a small sample volume at the required location and then processing the echoes to extract the Doppler-shifted frequencies.

Doppler imaging is often incorporated into real-time imaging systems, which provide electronic steering and focusing of a single acoustic beam and enables small volumes to be illuminated anywhere in the field-of-view (FOV) of the instrument. These locations can be visually identified on a two-dimensional B-mode image. A Fourier transform processor computes the Doppler spectrum backscattered from the sampled volumes, and by averaging the spectral components the mean frequency shift can be obtained. Typically, the calculated velocity is used to color code pixels in the B-mode image.

Despite the large variety of resources available for performing an echocardiogram or otherwise imaging the heart, it is difficult to accurately non-invasively identify the onset of a transplant rejection and it is difficult to discern characteristics of early transplant rejection without performing a biopsy. Therefore, it would be desirable to have a system and method for the early and non-invasive identification of heart transplant rejection and its symptoms.

### SUMMARY OF THE INVENTION

The present invention overcomes the aforementioned drawbacks by providing a system and method to non-invasively detect early cardiac rejection in heart transplant patients. It has been discovered that changes in the relationship between the native vasculature and ventricle wall mechanics occur in early cardiac rejection. Accordingly, the present invention provides a method for detecting cardiac rejection in a subject having a transplanted heart using a medical imaging apparatus. The method includes measuring a mechanical property of a ventricle wall in the transplanted heart with the medical imaging apparatus, measuring a property of the subjects's native vasculature with the medical imaging apparatus, and comparing the measured mechanical property of the ventricle to the measured property of the native vasculature. The method further includes generating a report indicative of a likelihood of cardiac rejection in the subject by matching a result of the comparison to a priori information acquired from subjects having transplanted hearts that experience cardiac rejection or subjects having transplanted hearts that do not experience cardiac rejection.

The invention is not limited to these aspects, and various other features of the present invention will be made apparent from the following detailed description and the drawings.

### BRIEF DESCRIPTION OF THE DRAWINGS

FIG. 1 is a block diagram of an ultrasonic imaging system that employs the present invention;

FIG. 2 is a block diagram of a transmitter which forms part of the system of FIG. 1;

FIG. 3 is a block diagram of a receiver that forms part of the system of FIG. 1;

FIG. 4A shows the relationship between a mechanical property of the aorta and a mechanical property of the left ventricle for a subject not experiencing cardiac rejection in accordance with the present invention;

FIG. 4B shows the relationship between a mechanical property of the aorta and a mechanical property of the left ventricle for a subject experiencing cardiac rejection in accordance with the present invention;

FIG. 5 is a flow chart setting forth the steps of a method for non-invasively determining a likelihood of transplant rejection in accordance with the present invention using the system of FIGS. 1-3; and

FIG. 6 is a radial map showing the output of a comparison of the torsional twists and turns of the aorta and cardiac ventricle in a subject with a transplanted heart undergoing early cardiac rejection in accordance with the present invention.

### DETAILED DESCRIPTION OF THE INVENTION

The present invention provides a method for identifying cardiac rejection based on measurements describing the motion of a transplanted heart and the motion of native structures within the transplant recipient. These measurements can be performed using a variety of medical imaging modalities, however, they can be performed efficiently and inexpensively using ultrasound. Thus, the following discussion relates primarily to the identification of cardiac rejection via ultrasound imaging.

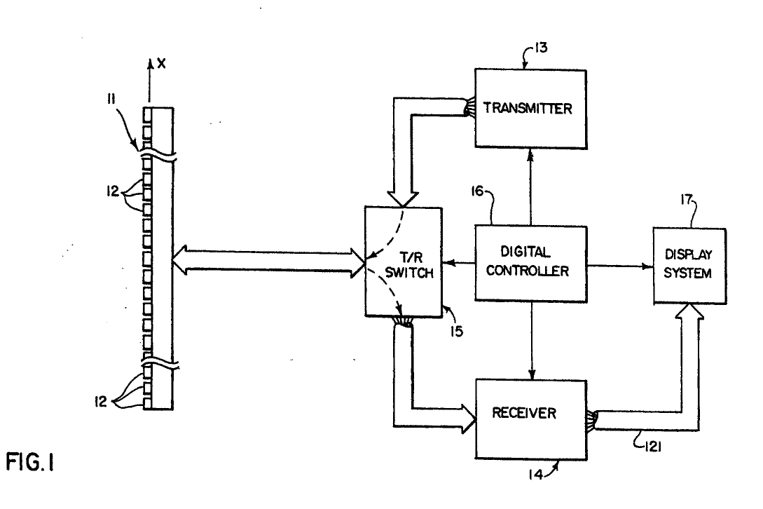

Referring particularly to FIG. 1, an ultrasonic imaging system includes a transducer array 11 comprised of a plurality of separately driven elements 12 which each produce a burst of ultrasonic energy when energized by a pulse produced by a transmitter 13. The ultrasonic energy reflected back to the transducer array 11 from the subject under study is converted to an electrical signal by each transducer element 12 and applied separately to a receiver 14 through a set of switches 15. The transmitter 13, receiver 14 and the switches 15 are operated under the control of a digital controller 16 responsive to the commands input by the human operator. A complete scan is performed by acquiring a series of echoes in which the switches 15 are set to their transmit position, the transmitter 13 is gated on momentarily to energize each transducer element 12, the switches 15 are then set to their receive position, and the subsequent echo signals produced by each transducer element 12 are applied to the receiver 14. The separate echo signals from each transducer element 12 are combined in the receiver 14 to produce a single echo signal which is employed to produce a line in an image on a display system 17.

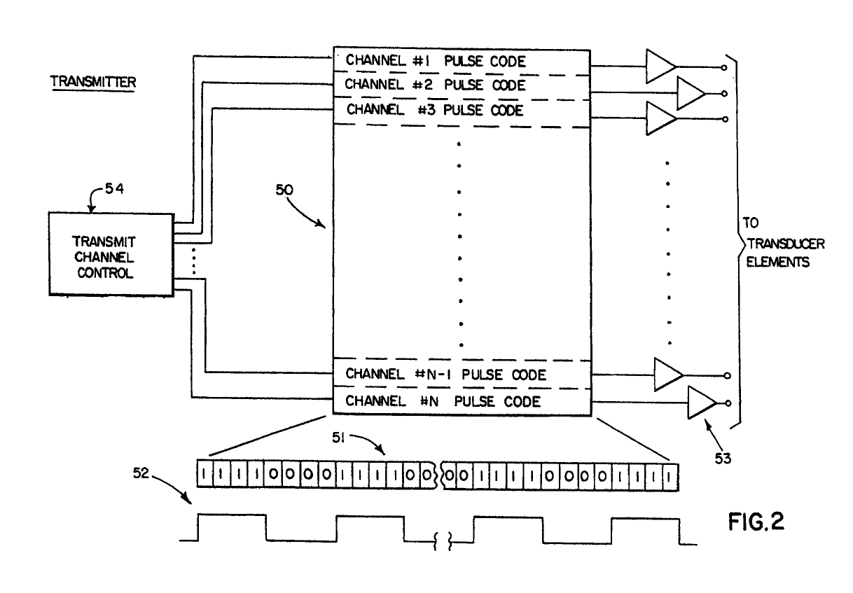

Referring particularly to FIG. 2, the transmitter 13 includes a set of channel pulse code memories which are indicated collectively at 50. Each pulse code memory 50 stores a bit pattern 51 that determines the frequency of the ultrasonic pulse 52 that is to be produced. This bit pattern is read out of each pulse code memory 50 by a master clock and applied to a driver 53 which amplifies the signal to a power level suitable for driving the transducer 11. In the example shown in FIG. 2, the bit pattern is a sequence of four “1” bits alternated with four “0” bits to produce a 5 MHz ultrasonic pulse 52. The transducer elements 11 to which these ultrasonic pulses 52 are applied respond by producing ultrasonic energy.

As indicated above, to steer the transmitted beam of the ultrasonic energy in the desired manner, the pulses 52 for each of the N channels must be produced and delayed by the proper amount. These delays are provided by a transmit control 54 which receives control signals from the digital controller 16 (FIG. 1). When the control signal is received, the transmit control 54 gates a clock signal through to the first transmit channel 50. At each successive delay time interval thereafter, the clock signal is gated through to the next channel pulse code memory 50 until all the channels to be energized are producing their ultrasonic pulses 52. Each transmit channel 50 is reset after its entire bit pattern 51 has been transmitted and the transmitter 13 then waits for the next control signal from the digital controller 16.

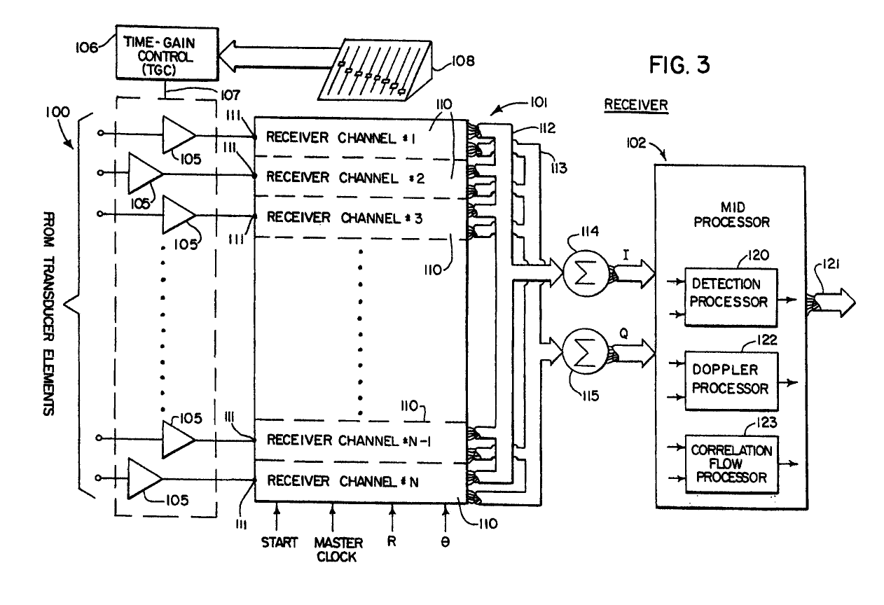

Referring particularly to FIG. 3, the receiver 14 is comprised of three sections: a time-gain control section 100, a beam forming section 101, and a mid processor 102. The time-gain control section 100 includes an amplifier 105 for each of the N receiver channels and a time-gain control circuit 106. The input of each amplifier 105 is connected to a respective one of the transducer elements 12 to receive and amplify the echo signal which it receives. The amount of amplification provided by the amplifiers 105 is controlled through a control line 107 that is driven by the time-gain control circuit 106. As is well known in the art, as the range of the echo signal increases, its amplitude is diminished. As a result, unless the echo signal emanating from more distant reflectors is amplified more than the echo signal from nearby reflectors, the brightness of the image diminishes rapidly as a function of range (R). This amplification is controlled by the operator who manually sets TGC linear potentiometers 108 to values which provide a relatively uniform brightness over the entire range of the scan. The time interval over which the echo signal is acquired determines the range from which it emanates, and this time interval is divided into segments by the TGC control circuit 106. The settings of the potentiometers are employed to set the gain of the amplifiers 105 during each of the respective time intervals so that the echo signal is amplified in ever increasing amounts over the acquisition time interval.

The beam forming section 101 of the receiver 14 includes N separate receiver channels 110. Each receiver channel 110 receives the analog echo signal from one of the TGC amplifiers 105 at an input 111, and it produces a stream of digitized output values on an I bus 112 and a Q bus 113. Each of these I and Q values represents a sample of the echo signal envelope at a specific range (R). These samples have been delayed in the manner described above such that when they are summed at summing points 114 and 115 with the I and Q samples from each of the other receiver channels 110, they indicate the magnitude and phase of the echo signal reflected from a point P located at range R on the ultrasonic beam.

Referring still to FIG. 3, the mid processor section 102 receives the beam samples from the summing points 114 and 115. The I and Q values of each beam sample is a digital number which represents the in-phase and quadrature components of the magnitude of the reflected sound from a point P. The mid processor 102 can perform a variety of calculations on these beam samples, where choice is determined by the type of image to be reconstructed. For example, if a conventional magnitude image is to be produced, a detection process indicated at 120 is implemented in which a digital magnitude M is calculated from each beam sample and output at 121.

M=\sqrt{I²+Q²}

The detection process 120 may also implement correction methods such as that disclosed in U.S. Pat. No. 4,835,689. Such correction methods examine the received beam samples and calculate corrective values that can be used in subsequent measurements by the transmitter 13 and receiver 14 to improve beam focusing and steering. Such corrections are necessary, for example, to account for the non-homogeneity of the media through which the sound from each transducer element travels during a scan.

The mid processor may also include a Doppler processor 122. Such Doppler processors often employ the phase information (φ) contained in each beam sample to determine the velocity of reflecting objects along the direction of the beam (i.e. direction from the transducer 11), where:

φ=tan⁻¹(I/Q).

The mid processor may also include a correlation flow processor 123, such as that described in U.S. Pat. No. 4,587,973, issued May 13, 1986 and entitled “Ultrasonic Method Can Means For Measuring Blood Flow And The Like Using Autocorrelation”. Such methods measure the motion of reflectors by following the shift in their position between successive ultrasonic pulse measurements.

The Velocity Vector Imaging (VVI) technique (Siemens Medical Solutions) can be used to analyze echocardiogram signals to determine cardiac tissue border movement in real time at any point in the cardiac cycle. VVI tracks motion using an integrated algorithm and can be used to calculate and display (in a color coded three-dimensional visual display known as a parametric map) ventricular wall mechanics, including quantities for wall shear, stress, and strain at any point of the ventricular wall. Recently, it has been demonstrated that VVI can also be used to visualize the wall mechanics of the aorta (see B. B. Kuersten, H. W. Rahmouni, J. C. Main, J. Davidson, M. G. St John Sutton, S. E. Wiegers, Novel Velocity Vector Imaging Ultrasound Technique Demonstrates Asymmetric Deformation in the Normal Aortic Root, Poster: 925-38 ACC 2006: Philadelphia/US).

The arterial-ventricular relationship and its effect on vessel wall mechanics and other vessel characteristics can be evaluated using echocardiogram measurements and VVI analysis of wall motion and deformation in the ventricle and in native vasculature such as the aorta. In healthy subjects, the wall mechanics patterns of the aorta and ventricle clearly show the typical helical pattern of the aorta and the twist and untwist of the ventricle. With aging, the walls of the native vasculature become stiffer and thicker. In transplant patients, the native vasculature age is different from the transplanted heart age. Thus, the relationship between the wall mechanics patterns of the ventricle and certain properties of the native vasculature is somewhat different in heart transplant patients than it is in normal patients. Because the difference between the native vasculature age and the transplanted heart age applies to the native vasculature as a whole rather than being limited to the native aorta, the wall mechanics of the ventricle can be compared to a number of different age-related properties from any part of the native vasculature. Thus, the present invention is not limited to comparisons of the wall mechanics patterns of the ventricle and the aorta.

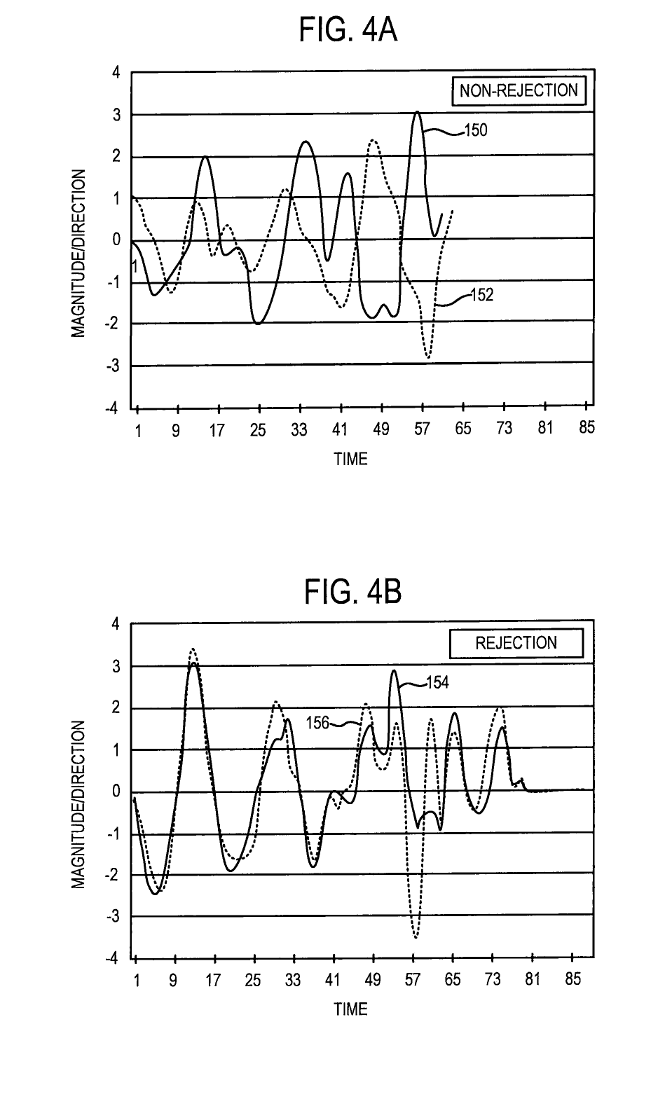

Referring to FIG. 4, the present invention characterizes the relationship between the wall mechanics patterns of the ventricle and certain properties of the native vasculature for heart transplant patient undergoing early cardiac rejection and heart transplant patients whose hearts are functioning normally. In particular, it is observed that this relationship is much different and far less organized for patients undergoing cardiac rejection. For example, such relationships can be seen for a heart transplant patient without cardiac rejection in FIG. 4A and for a heart transplant patient experiencing cardiac rejection in FIG. 4B. For the patient not experiencing cardiac rejection, the displacement of the aorta 150 is relatively out-of-synch with the displacement of the left ventricle 152. In contrast, for the patient experiencing cardiac rejection, the displacement of the aorta 154 is visibly synched with that of the left ventricle 156. As will be described in detail below, the present invention utilizes the above-described systems to measure and analyze the wall mechanics of the transplant patient's ventricle, measure certain properties of the native vasculature, compare the wall mechanics of the ventricle with those properties of the native vasculature, output the result of the comparison, and match the outputted result either to the results of similarly-obtained comparisons from heart transplant patients whose hearts are undergoing early rejection or to results of similarly-obtained comparisons from heart transplant patients whose hearts are functioning normally. If these results more closely match the results of similarly-obtained comparisons from heart transplant patients whose hearts are undergoing early rejection, then it is indicated that the transplanted heart may be undergoing early rejection.

“Wall mechanics” refers to calculated values indicative of the mechanical properties of the tissue, such as wall shear, wall stress, and wall strain at specific locations on the ventricle or vasculature wall tissue as calculated and recorded continually in real time. In “real time” means that calculations are done for each reconstructed image frame. “Wall shear” is the force acting parallel to the surface of the wall tissue caused by the movement of blood or other fluid through a blood vessel or cardiac ventricle and “wall stress” is the pressure exerted per unit cross-sectional tissue wall area for a defined cross-section of the cardiac ventricle or blood vessel. Likewise, “wall strain” is a measure of the deformation of a given tissue wall section relative to its original length so that a tissue wall that is stretched beyond its normal length has a positive wall strain, while a tissue wall that is contracted to a length shorter than its normal length has a negative wall strain.

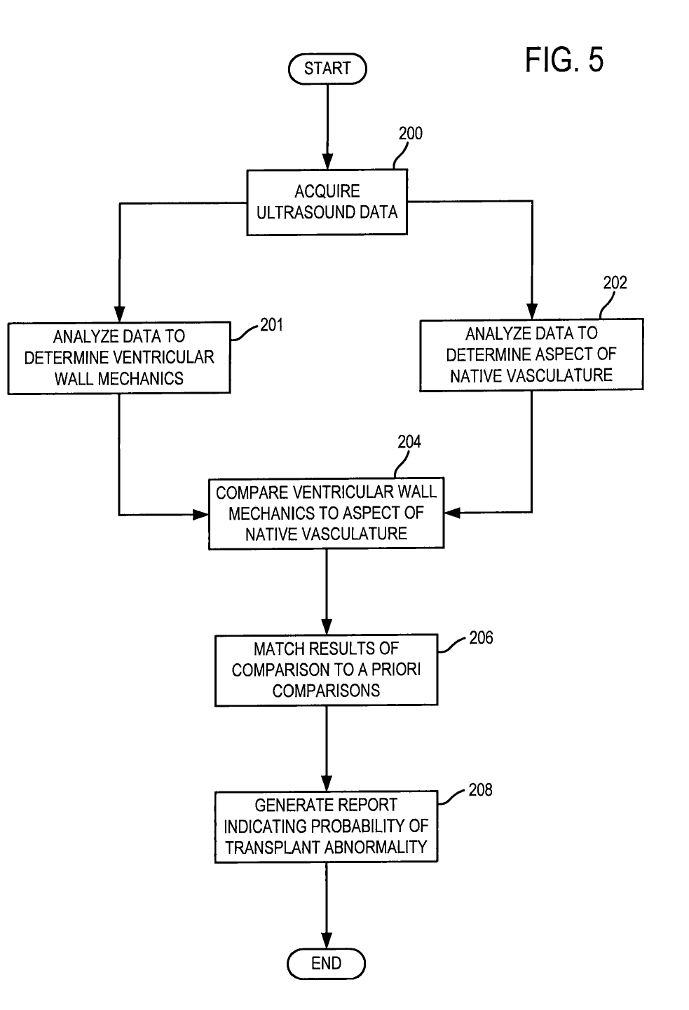

Referring to FIG. 5, a method for detecting cardiac rejection in accordance with the present invention begins at process block 200 with the acquisition of ultrasound data corresponding to ventricular wall mechanics and at least one property of the native vasculature. Conventional 2D or 3D ultrasound techniques may be used acquire this data. Properties of the native vasculature that may be measured include the wall mechanics of the aorta, a cross-sectional area of a blood vessel, the thickness of vascular walls, and the velocity of blood flowing through a blood vessel, which can be determined using Doppler imaging techniques. It is contemplated that these measurements and other acquired ultrasound data are collected, processed, and stored for offline analysis.

It should be noted that measuring a property of the native vasculature is not limited to imaging the area around the heart. For example, another property of the native vasculature that can be measured by ultrasound is the intima-media thickness (IMT). The intima and the media are the two inner layers of the arterial wall and they generally thicken with age. Thus, an IMT test can determine apparent vascular age and health. Intimal thickening is a complex process that depends on a variety of factors, including changes in shear stress and blood pressure. Because the carotid artery of the neck is more easily accessible by an ultrasound probe, IMT tests are typically performed on the carotid artery (CIMT), rather than the vasculature near the heart. The most common CIMT test uses an FDA-approved software program, SonoCalc, obtains CIMT measurements for many different points along each carotid artery to determine an average thickness. Therefore, it may be advantageous to perform an IMT test on the ceratoid artery of the neck rather than the vasculature near the heart when measuring this property of the native vasculature.

At process block 201, the acquired ultrasound data is analyzed to quantify ventricular wall mechanics. This can be performed using motion tracking or ultrasound analysis software such as AxiUS™ “Velocity Vector Imaging (VVI),” which is made and distributed by Siemens Medical Solutions. VVI uses an angle independent algorithm to evaluate strain and velocities and it performs whole heartbeat analysis using Fourier techniques and constraints on the global coherence of the tracked geometry. VVI first tracks reference points and then scales the motion of adjacent points based on the observed motion of the reference points. At a second order of refinement, VVI tracks the tissue/cavity border using local correlations and snake contouring. At a third order of refinement, image speckles are tracked along the direction of the border. At each level, tracking is initially done over a 2 cm band and then refined over successively smaller bands down to 5 pixels. The VVI software allows the points of interest to be manually set. For example, in analyzing ventricular wall mechanics, one point of interest may be a location in the cardiac left ventricle wall.

The tracking algorithm is automatically applied to a set of points on a contour in a sequence of two dimensional sequences of B-mode images. The velocity is displayed as a vector overlaid on the B-mode image. The length and direction of the tracking arrows reflects the magnitude and direction of the velocity vector at that point. Application of this method to an entire R-R interval provides a “real time” display of cardiac tissue motion. Preferably, VVI is used to convert three-dimension real time images into detailed wall mechanics data, including measurements of stress, strain, and wall shear.

At process block 202, the acquired ultrasound data is analyzed to quantify a property of the native vasculature. For example, aortic wall mechanics can be quantified from the acquired ultrasound data using ultrasound analysis software as discussed above for process block 201. Data corresponding to other properties of the native vasculature, such as wall thickness or cross-sectional area, can also be analyzed and quantified at step 202.

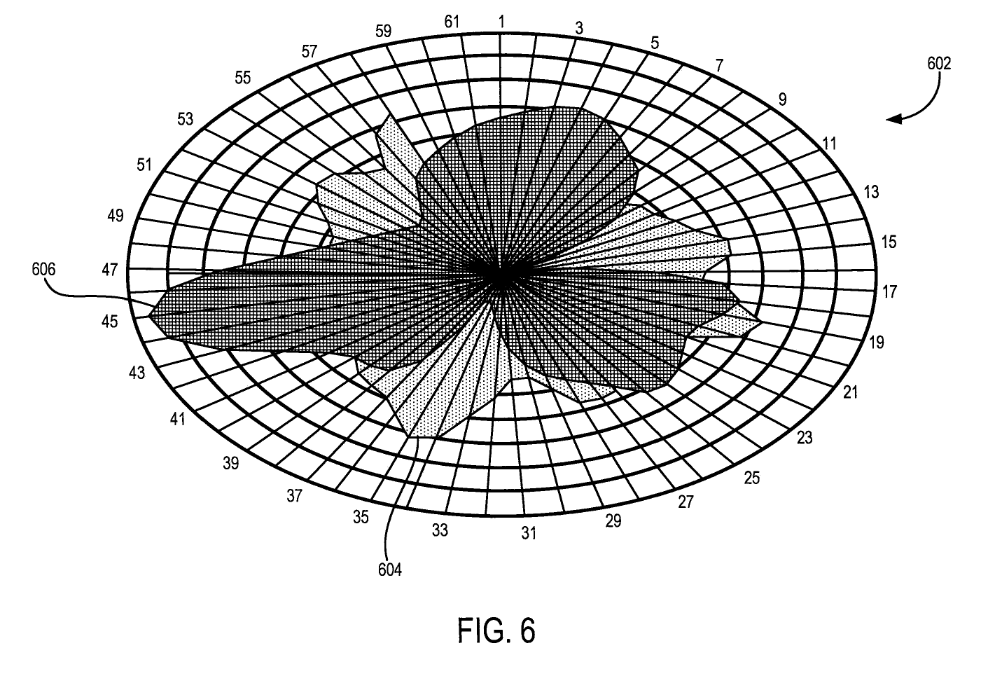

Referring now to FIGS. 5 and 6, thereafter, the wall mechanics data of the ventricle wall and the data from the chosen property of the native vasculature are compared at process block 204. Although there are a number of ways the results of this comparison may be outputted, it is contemplated that they are outputted to a three-dimensional, color-coded parametric map displaying the compared ventricle wall mechanics and native vasculature measurement patterns. In such a map, gradients of color are mapped to values or parameters, such as rotation velocity, direction of motion, or wall mechanics values as calculated by VVI. Colors may be used to indicate the locations of the tissues being compared. The parametric map may further be overlaid on an anatomical display of the heart and great vessels. It is also contemplated that the results of the comparison are outputted to a three-dimensional, color-coded bubble chart displaying the compared ventricle wall mechanics and native vasculature measurement patterns. In such a chart, gradients of color are mapped to values showing the torsional twists of the compared tissues. Colors may be used to indicate the locations of the tissues being compared. For example, FIG. 6 provides a radial map 602 for a subject experiencing post-transplant cardiac rejection that shows the torsional twists and turns of the subject's aorta and left ventricle, as indicated at 604 and 606, respectively. In this case, the direction of the torsional motion is depicted in the angular direction and ranges from 1 to 61 degrees, while the magnitude of this motion is depicted in the radial direction and ranges from −2.5 at the map's center to 2.0 in its periphery. These motion patterns 604, 606 have reduced symmetry compared to those of subjects not experiencing cardiac rejection and the aortic twist pattern 604 is not strongly correlated with the ventricular twist pattern. Thus, it is sometimes possible to identify cases of cardiac rejection by visual inspection of such maps.

Referring again to FIG. 5, at process block 206, the outputted results of the comparison are evaluated against similarly-obtained comparison outputs from other transplant patients, including both those who did and did not experience early cardiac rejection. The comparison outputs can, for example, be obtained from a reasonable sample size of patients in both prospective groups, that is, those who experienced cardiac rejection and those who did not. When the outputted results of the comparison are evaluated against those of the two groups, the outputted results will more closely correlate to the outputted results of one of the prospective groups. This match indicates whether the patient is experiencing early cardiac rejection. A number of different methods can be used to evaluate and match the outputs, including without limitation visual inspection or using analysis software. Accordingly, a report indicating the subject's risk of cardiac rejection is generated at process block 208.

The present invention therefore allows the early identification of post-transplant cardiac rejection by analyzing relationships between a property of a subject's native vasculature and the ventricular wall mechanics of the transplanted heart. These relationship patterns are fundamentally different for patients with normally functioning transplanted hearts versus patients undergoing early cardiac rejection. Specifically, the relationship patterns in patients undergoing early cardiac rejection show an identifiable pattern of disorganization over patients whose transplanted hearts are functioning normally.

The present invention has been described in terms of preferred embodiments, and it should be appreciated that many equivalents, alternatives, variations, and modifications, aside from those expressly stated, are possible and within the scope of the invention. Therefore, the invention should not be limited to a particular described embodiments.

What is claimed is:

1. A method for detecting cardiac rejection in a subject having a transplanted heart using a medical imaging apparatus, the method comprising the steps of:
a) measuring a mechanical property of a ventricle wall in the transplanted heart with the medical imaging apparatus;
b) measuring a property of the subjects's native vasculature with the medical imaging apparatus;
c) comparing the measured mechanical property of the ventricle to the measured property of the native vasculature; and
d) generating a report indicative of a likelihood of cardiac rejection in the subject by matching a result of the comparison performed in step c) to a priori information acquired from at least one of subjects having transplanted hearts that experience cardiac rejection and subjects having transplanted hearts that do not experience cardiac rejection.

2. The method of claim 1 wherein the medical imaging apparatus is an ultrasound device.

3. The method of claim 2 wherein measuring the mechanical property of the ventricle wall is at least one of stress, shear, and strain.

4. The method of claim 3 wherein the measured property of the native vasculature is a mechanical property of a vascular wall.

5. The method of claim 4 wherein the mechanical property of the vascular wall is at least one of stress, shear and strain.

6. The method of claim 5 wherein the vascular wall is an aortic wall.

7. The method of claim 6 wherein steps a), b), and c) include employing motion tracking to measure and compare the mechanical properties of the aortic wall and ventricle wall.

8. The method of claim 3 wherein the measured property of the native vasculature is a thickness of a vascular wall.

9. The method of claim 8 wherein the thickness of the vascular wall is measured in a carotid artery of the subject using a carotid intima-media thickness test.

10. The method of claim 1 wherein the ventricle wall is a left ventricle wall.

11. The method of claim 10 wherein the mechanical property of the left ventricle wall is measured at a mid-cavity of the ventricle.

12. The method of claim 1 wherein step c) further includes outputting the results of the comparison as a visual display.

13. The method of claim 12 wherein the visual display is at least one of a parametric map and a bubble chart.

14. The method of claim 13 wherein the parametric map is color-coded.

15. The method of claim 14 wherein the color-coded map further includes three-dimensional indicators of wall shear, stress, and strain.

16. The method of claim 14 wherein the visual display further includes an anatomical representation of the heart and great vessels.
